(19) United States
(12) Patent Application Publication
(10) Pub. No.: US 2010/0130856 A1
(43) Pub. Date: May 27, 2010

(54) METHOD AND APPAARATUS FOR THE ELATOGRAPHIC EXAMINATION OF TISSUE

(76) Inventors: Ingolf Sack, Berlin (DE); Jürgen Braun, Berlin (DE)

Correspondence Address: FOLEY AND LARDNER LLP;SUITE 500, 3000 K STREET NW, WASHINGTON, DC, 20007, US

(21) Appl. No.: 12/451,278

(22) PCT Filed: May 7, 2008
(86) PCT No.: PCT/EP2008/055617

§ 371 Date: Nov. 4, 2009

(30) Foreign Application Priority Data
May 8, 2007 (DE) 10 2007 022 469.0

(51) Int. Cl. A61B 8/00

(52) U.S. Cl. 600/438

(57) ABSTRACT

The invention relates to a method for elastographically examining tissue. Said method comprises the following steps: at least one mechanical wave that oscillates primarily or exclusively perpendicular to the direction of propagation thereof is excited in the tissue (31) which has first elastic properties at least at one first point in time and second elastic properties differing from the first elastic properties at least at one second point in time; and a first deflection or deflection rate of an oscillation of the wave is determined at the first point in time as a measure of the first elastic properties, and a second deflection or deflection rate of an oscillation of the wave is determined at the second point in time as a measure of the second elastic properties. The invention further relates to an apparatus for elastographically examining tissue.

The invention relates to a method for the elastographic examination of tissue with the features as per claim 1 and an apparatus with the features as per claim 25.

The importance of elasticity (to be precise: shear elasticity or shear modulus, shear strength etc.) in assessing the health of an organ has been known for centuries. For example, manual touching of the breast for cancer prevention is often more sensitive than the application of modern imaging methods. Likewise, liver dysfunction is connected to a change in the elasticity which precedes unambiguous morphological (i.e. visible in MRI) changes.

Elastography was developed in recent years in order to be able to use the high sensitivity of the shear modulus for pathology. The basic principle of all current elastographic techniques is touching the tissue with a defined stress (i.e. force per unit area) and recording the distortion response in the tissue by means of imaging. Shear wave elastography was developed for “scanning” deeper-lying and shielded types of tissue, the clinical relevance of which could be demonstrated for the diagnosis of breast tumors and liver cirrhosis.

Recently, there have been attempts in cardiac elastography to utilize the heart beat as a mechanical stimulus of the myocardial deformation and thus measure elastic parameters in the living heart. The force with which the myocardium deforms was estimated semi-empirically for the analysis of the measured distortion data in the articles “Myocardial elastography—a feasibility study in vivo”, Konofagou E E, D'Hooge J, Ophir J., Ultrasound Med Biol 2002; 28(4):475-482 and “Single Breath Hold Transient MR-Elastography of the Heart—Imaging Pulsed Shear Wave Propagation induced by Aortic Valve Closure”, Sinkus R, Robert B, Gennisson J-L, Tanter M, Fink M, Proc 14th Annual Meeting ISMRM. Seattle. 2006. p 77.

The object to be achieved by the present invention consists of creating a method and an apparatus for the elastographic examination of tissue with temporally changing elastic properties, which afford improved determination of the elastic properties of the tissue.

This object is achieved by the method having the features as per claim 1 and by the apparatus having the features as per claim 15. Developments of the invention are specified in the dependent claims.

Accordingly, provision is made for a method for the elastographic examination of tissue, comprising the following steps:
exciting at least one mechanical wave in the tissue, which wave oscillates predominately or exclusively transversely with respect to the direction of propagation thereof, wherein
the tissue has first elastic properties at least a first point in time and second elastic properties at least a second point in time, which second elastic properties differ from the first elastic properties; and
a first deflection or deflection rate of an oscillation of the wave is determined at the first point in time as a measure of the first elastic properties and
a second deflection or deflection rate of an oscillation of the wave is determined at the second point in time as a measure of the second elastic properties.

In this method, a shear wave, i.e. a wave which oscillates predominately or exclusively transversely with respect to the direction of propagation thereof, is coupled into the tissue to be examined. In order to draw conclusions regarding the elastic properties (e.g. shear modulus) of the tissue, the deflection and/or deflection rate (e.g. transversely with respect to the direction of propagation of the wave) with which sections of the tissue oscillate due to the coupled-in wave is determined at least two points in time. This can be effected in the same section of the tissue for each of the two points in time or, for example, in different sections as well, which sections have comparable elastic properties and a comparable time profile of the elastic properties.

Of course, it goes without saying that a number of shear waves can be coupled-in which for example superpose in the tissue to be examined. The shear waves are generated by means of an excitation unit which is outside of the tissue, i.e. the wave is not generated by tensing or relaxing of the tissue itself.

A biological (in particular human or animal) tissue is considered as a tissue. In particular, the tissue is a myocardial tissue which has elastic properties which vary in time depending on the heart beat, wherein, for example, it has first elastic properties during systole and second first elastic properties during diastole.

The deflection or deflection rate of the tissue shear waves can be determined not only at the first and at the second point in time, but, moreover, at additional points in time. For example, a time interval during which the tissue has the first or the second elastic properties can be measured. If the elastic properties of the tissue change periodically, a (first and/or second) deflection or deflection rate can moreover be determined repeatedly, wherein the repeated determination is effected with the period with which the elastic properties change. The majority of the (first and/or second) values can be respectively averaged in order to obtain an averaged first and/or an averaged second deflection or deflection rate.

In one variant of the invention, the first and second deflection or deflection rate of the excited wave is determined by ultrasound and/or magnetic resonance imaging. By using magnetic resonance imaging in particular, it is possible to detect the components of an oscillation of the wave, i.e. the deflection or the deflection rate, separately in different spatial directions. However, the method according to the invention also comprises the variant in which the resultant of the oscillation is measured directly. In particular, it is also possible that only one component of the deflection or deflection rate is measured. The cross-correlation method or the Doppler method can, inter alia, be used as ultrasound variants.

In another development of the invention, in at least one further section of the tissue, a further first deflection or deflection rate is determined at a point in time at which the tissue has the first elastic properties, and a further second deflection or deflection rate is determined at a point in time at which the tissue has the second elastic properties. In other words, the measurement is performed resolved not only in time but also in space. The further first and the further second deflection or deflection rate can be determined at the same time as the first and the second deflection or deflection rate is determined. In another variant, the further first and the further second deflection or deflection rate are determined offset in time to determining the first and the second deflection or deflection rate.

In a further refinement of the method according to the invention, a first and the second deflection are determined in the form of a first and second amplitude of the deflection of the oscillation or of the deflection rate of the oscillation. In particular, the time profile of the deflection and the deflection rate can in each case be a harmonic function, and the deflection and deflection rate can be phase-shifted with respect to one another.

At least a first and a second elastic parameter of the tissue can be determined on the basis of the determined first and second deflection, for example in the form of a first and second amplitude. A possibility for determining an elastic parameter (of the shear modulus) on the basis of the determined first and second amplitude results from the following observations, wherein (1) the total energy balance of an elastic deformation which consists of kinetic energy and strain energy is established, (2) the energy flux through a unit surface per unit time is derived, (3) an elastic wave which is harmonic in time is assumed as a deflection function, which wave passes through a medium at two different times with different elasticities and (4) the ratio of the wave amplitudes at points 1 and 2 in time at different elasticities are derived assuming a constant energy flux.

The propagation of an elastic wave in a medium is connected to the transport of energy. The change in the total energy E in a deformed elastic body surrounded by a volume V is given by the time profile of the kinetic and the potential energy (the strain energy), that is to say (using Einstein's summation convention):

$$E = {E}_{kin} + {E}_{pot} \\ {E}_{kin} = \frac{1}{2} {\int }_{V} ρ {\dot{u}}_{i} {\dot{u}}_{i} d V ; \\ {E}_{pot} = \frac{1}{2} {\int }_{V} {c}_{ijkl} \frac{\partial {u}_{i}}{\partial {x}_{j}} \frac{\partial {u}_{k}}{\partial {x}_{l}} d V \qquad ( 1. )$$

Here, x refers to the position, u refers to the vector field of the displacement and cᵢⱼₖₗ refers to the components of the elasticity tensor. ρ is the density, assumed to be 1 kg/1 in the myocardium. The change in total energy is given by

$$\frac{d E}{d t} = {\int }_{V} ρ {\dot{u}}_{i} {\ddot{u}}_{i} + {c}_{ijkl} \frac{\partial \dot{{u}_{i}}}{\partial {x}_{j}} \frac{\partial {u}_{k}}{\partial {x}_{l}} d V = - {\int }_{S} {F}_{j} d {S}_{j} . \qquad ( 2. )$$

Here, the right-hand side of equation 2 represents the energy flux through a surface with the normal nⱼ. After applying the product rule to the strain energy term in equation 2 and Gauss's law, this results in

$${F}_{j} = - {c}_{ijkl} {\dot{u}}_{i} \frac{\partial {u}_{k}}{\partial {x}_{l}} = - {σ}_{ij} {\dot{u}}_{i} . \qquad ( 3. )$$

This holds for deformed materials in force equilibrium, excluding gravity. The direction and magnitude of the energy density flux vector F specify the direction of the energy flux and the magnitude of the energy flowing per unit time through a unit surface with the normal vector n. In an isotropic elastic material, the following holds for F with Lame parameters λ and μ:

$${F}_{j} = - λ \frac{\partial {u}_{k}}{\partial {x}_{k}} {δ}_{ij} {\dot{u}}_{i} - 2 μ \frac{\partial {u}_{i}}{\partial {x}_{j}} {\dot{u}}_{i} . \qquad ( 4. )$$

The propagation of a planar elastic wave is determined by three eigenmodes M which, relative to n, propagate as a longitudinal mode (L) and transverse modes (T) with the phase speed c_{M}.

ρc_{L}²=(λ+2μ)n²

ρc_{T}²=2μn² (5.)

In the isotropic case, the two transverse modes are degenerate. The direction of polarization relative to n is given by the corresponding eigenvector U_{M} which coincides with the Cartesian unit vector eᵢ if n lies on the axis of the elastic reference frame. In order to evaluate the energy flux in time harmonic elastography, planar wave modes with amplitude A_{M} and angular frequency ω are assumed:

$${u}_{M} = {A}_{M} {U}_{M} exp ( iω [ x \cdot \frac{n}{{c}_{M}} - t ] ) . \qquad ( 6. )$$

Reference is made to the fact that for harmonic waves the energy flux corresponds to an infinite succession of pulses propagating with the group velocity ∂c_{M}/∂n. Substituting equation 6 into equation 4 results in the vector components F_{L} and F_{T}, which constitute the energy density flow parallel and perpendicular to the normal wave vector:

|F_{M}|=c_{M}A_{M}²ω² M=T, L. (7.)

Hence, |F_{M}| is constant when excited by propagating harmonic planar waves in space and time. If two wave amplitudes A_{1M} and A_{2M} are observed at two points in time during the cardiac phase, their ratio to one another corresponds to the relative change in the wave speed on the basis of elasticity changes in the myocardium:

$${R}_{M} = {( \frac{{A}_{1 M}}{{A}_{2 M}} )}^{2} = \frac{{c}_{2 M}}{{c}_{1 M}} . \qquad ( 8. )$$

In shear-wave-based elastography, the assumption of incompressibility of soft biological tissue has been established. Using this restriction, λ is infinite and R_{L} equals one, i.e. there is no change in the wave amplitude as a result of compression waves. By contrast, the convergent shear modulus μ results in a change in the amplitude to the fourth power:

$${R}_{T} = {( \frac{{A}_{1}}{{A}_{2 }} )}^{4} = \frac{{μ}_{2}}{{μ}_{1}} . \qquad ( 9. )$$

In a further variant of the invention, the tissue has the shear modulus μ₁ at the first point in time and the shear modulus μ₂ at the second point in time. Their ratio thereof with respect to one another is determined on the basis of a first amplitude A₁ determined at the first point in time and the second amplitude A₂ determined at the second point in time according the above equation (9). Reference is made to the fact that this is of course not restricted to the myocardium but can be applied to all tissues which have time-varying elastic properties, e.g. a different muscular tissue.

In a further variant of the invention, a first and the second amplitude of the wave is in each case determined by a Fourier transformation or a correlation of the deflection or deflection rate with a harmonic oscillator function. By way of example, the harmonic oscillator function has an oscillation frequency which corresponds to the frequency with which the wave is excited in the tissue. By forming the correlation, the deflection signal of the excited wave can be separated from an intrinsic motion of the tissue (e.g. contraction and extension of a muscle, e.g. of the myocardium) and hence the amplitude (of the deflection or else of the deflection rate) of the oscillation can be determined in a filtered fashion.

In the following text, forming the correlation is considered in the case where the wave excited in the tissue is detected by magnetic resonance. Exciting a wave in the tissue and detecting the wave using magnetic resonance is referred to as magnetic resonance elastography (MRE). In this example, a characteristic time-dependent phase signal φ(t) is determined for the wave and a deflection rate \dot{u}(t) of an oscillation of the wave is calculated from the derivative of said phase signal with respect to time \dot{φ}. The deflection rate \dot{u}(t) in turn is correlated with a complex harmonic function which has the same frequency and this results in the following time profile of the wave amplitude:

$$A ( t ) = \frac{1}{N π} \vert {\int }_{t}^{t + Δ t} \dot{u} exp ( iω τ ) d τ \vert ;$$

$$with$$

$$Δ t = N \cdot \frac{2 π}{ω} , N = 1 , 2 , 3 , …$$

The integration increment Δt is for example selected such that the deflection amplitude A(t) is determined over N complete wave cycles, i.e. A(t) has an improved time resolution which has been shortened by N times the number of sampling points of a vibration cycle. Instead of the deflection rate \dot{u}(t), it is also possible to correlate a deflection u(t) determined from the phase signal with the harmonic function in order to determine the amplitude.

Moreover, the invention relates to an apparatus for the elastographic examination of tissue, with
deflection determination means (4) for determining a deflection and/or deflection rate of mechanical waves excited in the tissue (31) which oscillate predominately or exclusively transversely with respect to the direction of propagation thereof, wherein
the tissue (31) has first elastic properties at a first point in time and second elastic properties at a second point in time, which second elastic properties differ from the first elastic properties; and
the deflection determination means (4) are designed and provided to determine a first deflection or deflection rate at the first point in time and a second deflection or deflection rate at the second point in time.

In principle, the deflection determination means can have any design, e.g. they can be based on ultrasound or magnetic resonance. In particular, the deflection determination means can comprise a programmable unit with control and evaluation software which implement e.g. the above-described methods for correlating a deflection or deflection rate signal, or, in general, the procedures for determining a deflection or deflection rate signal, i.e. detection and evaluation of a signal characteristic of the deflection or deflection rate.

Furthermore, the apparatus can have wave excitation means for exciting at least one mechanical wave in the tissue. Examples of such wave excitation means are described in the German patent application 10 2006 037160.7. Reference is made to the fact that the deflection determination means can be designed separately from the wave excitation means and, for example, can also be provided to interact with different wave excitation means.

The invention will be explained in more detail in the following text on the basis of exemplary embodiments and with reference to the figures, in which:

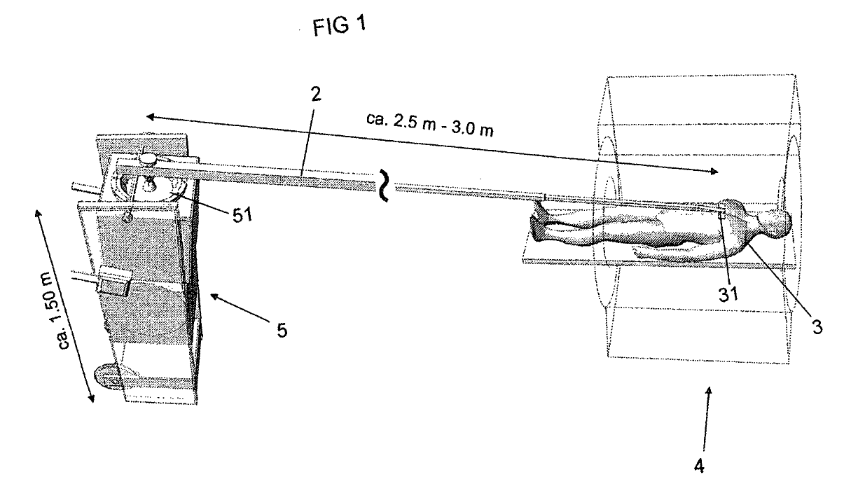

FIG. 1 shows a variant of an MRE apparatus;

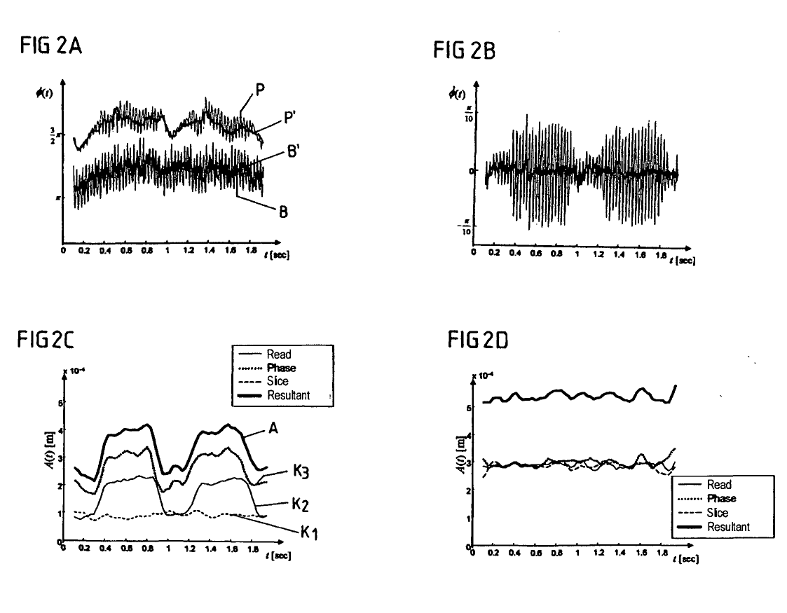

FIGS. 2a to 2d show evaluations of an MRE experiment in the myocardium and in the thorax of a test subject;

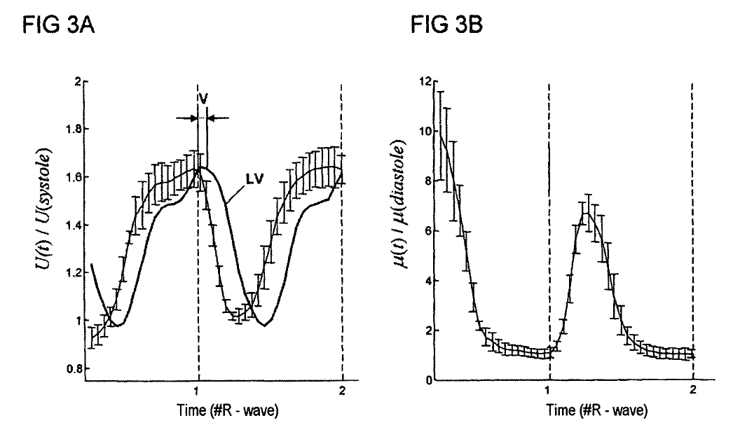

FIGS. 3a and 3b show an evaluation of a further MRE experiment in the myocardia of six test subjects.

FIG. 1 shows an MRE apparatus as can be used to carry out the method according to the invention. The apparatus comprises wave generation means 5 which generate mechanical oscillations by means of a loudspeaker membrane 51. The oscillations generated by the loudspeaker membrane 51 are transmitted to a test subject 3 via a rod-shape transmission element 2 and are coupled into the tissue 31 to be examined of the test subject 3. The mechanical waves excited in the tissue 31 by this are detected by means of deflection determination means in the form of an MRI scanner 4 and a deflection or deflection rate of the excited waves is determined.

In a development, the transmission element is coupled to a couch or a seating device on which the test subject is positioned during the measurement and said element transmits the oscillations onto the couch or seating device. The tissue to be examined of the test subject is finally excited by the oscillating couch or seating device. In one variant, the loudspeaker membrane is integrated into the couch or seating device in order to oscillate the latter and so the transmission element can be dispensed with.

FIGS. 2a to 2d relate to evaluations of an MRE measurement according to the invention in the myocardium and the thorax of a test subject. Mechanical waves were coupled into the myocardial tissue or into the thorax of the test subject and detected by magnetic resonance. FIG. 2a plots the phase signal of the magnetic resonance measurement (ordinate) which is characteristic of the deflection of an oscillation of the wave against time (abscissa) for the myocardium (curves P) and the thoracic cage (curve B). For comparison, a measurement curve P′ and B′ is also illustrated in each case, which curves were recorded without mechanical excitation of the tissue. The measurements were undertaken for approximately two cardiac phases.

It can be seen in FIG. 2a that the amplitude of the phase signal φ of the myocardium measurement with mechanical wave excitation clearly changes in time, whereas the amplitude of the phase signal of the waves excited in the thoracic cage is basically constant.

FIG. 2b relates to the myocardium measurement from FIG. 2a, wherein the phase signal was filtered by rather than using the pure phase signal φ, the time derivative \dot{φ} thereof was used and as a result of this the amplitude modulation occurring over the cardiac phase is illustrated even more clearly.

The phase signal of the magnetic resonance can be converted into wave amplitudes by, as described above, correlating the phase signal with a harmonic function with the same frequency as the oscillations excited in the tissue. After performing such a correlation, the curve profiles illustrated in FIG. 2c for the time dependence of the oscillation amplitude of the waves excited in the myocardium result, wherein the amplitudes of the three spatial components of the MRI measurement (slice gradient, readout gradient and phase encoding direction, curves K₁, K₂, K₃) and the magnitude A of the resultant of the oscillation are illustrated. The curve K₁ was recorded in a direction parallel to the direction of propagation of the coupled-in wave. However, due to its transverse nature, the wave does not have or only has a very small oscillation component in this direction, and so, in principle, the amplitude in this direction correspondingly has no time dependence.

The profile of the wave amplitude for the other spatial directions (curves K₂, K₃) corresponds to the profile of the amplitude of the phase signal (FIGS. 2a, 2b). The wave amplitudes vary over the cardiac phase, with a higher amplitude occurring when the myocardium is relaxed, i.e. has a lower stiffness than in the tensed state of the myocardium. More precisely, FIG. 2c shows that the wave amplitude in the region of the early systole (at t=1-1.1 s) falls to approximately half the value compared to the diastole, which allows the conclusion that there is an approximately 16-fold increase in the elasticity of the myocardium during this phase of the heart beat.

In the case of the measurements illustrated in the FIGS. 2a to 2c, 360 MRI images were recorded in each case, with six records being made per cycle of the mechanical waves excited in the tissue. Here, the integration increment Δt when performing the correlation was selected such that the wave amplitude was determined over a complete cycle of the mechanical wave, i.e. an oscillation duration, which is why the time resolution of the correlation signal (of the amplitude in FIG. 2c) is improved compared to the phase signal.

FIG. 2d shows an evaluation of the phase signal of the thoracic cage measurement which is analogous to FIG. 2c. The resultant amplitude signal basically has no time dependence.

FIGS. 3a and 3b relate to measurements of the myocardia of six test subjects. FIG. 3a plots the average amplitude of the mechanical oscillations of the test subject respectively excited in the myocardium (ordinate) over time (abscissa). Moreover, the diameter LV of the left heart ventricle is illustrated (dashed line) and this makes possible a comparison of the time profile of the amplitude A and the time profile of the heart morphology (heart volume). The error bars correspond to the standard deviation between individuals.

It can be seen that the amplitude signal A drops significantly during the systole. More precisely, the drop in the wave amplitude precedes the drop in the ventricle volume (by approximately 60 ms). This makes it possible to conclude that tensing the myocardium begins directly with the arrival of the R-wave (at the end of the diastole), wherein the heart volume remains constant over a period of time V after the start of the contraction of the myocardium (isovolumetric contraction phase).

FIG. 3b shows an evaluation of the amplitude from FIG. 3a, wherein the time profile of the shear modulus is illustrated in relation to the shear modulus of the myocardium during the diastole (ordinate). It can be seen that the elasticity modulus p increases during the systole—unlike the amplitude—and this is due to the contraction of the myocardium during this cardiac phase.

### LIST OF REFERENCE SIGNS

2 Transmission element
3 Test subject
31 Tissue
4 MRI scanner
5 Wave excitation means
51 Loudspeaker membrane

1. A method for the elastographic examination of tissue, comprising the following steps:
exciting at least one mechanical wave in the tissue, which wave oscillates predominately or exclusively transversely with respect to the direction of propagation thereof, wherein
the tissue has first elastic properties at least a first point in time and second elastic properties at least a second point in time, which second elastic properties differ from the first elastic properties; and
a first deflection or deflection rate of an oscillation of the wave is determined at the first point in time as a measure of the first elastic properties and
a second deflection or deflection rate of an oscillation of the wave is determined at the second point in time as a measure of the second elastic properties.

2. The method as claimed in claim 1, characterized in that the first and second deflection or deflection rate is determined by ultrasound and/or magnetic resonance imaging, and the tissue in which the wave is excited is a myocardial tissue which has elastic properties which vary in time depending on the heart beat.

3. (canceled)

4. (canceled)

5. (canceled)

6. The method as claimed in claim 1, characterized in that the first and the second deflection rate are determined in the form of a first and second amplitude of the deflection rate of the oscillation.

7. (canceled)

8. The method as claimed in claim 1, characterized in that the first and the second deflection or deflection rate is in each case determined separately for all components of the oscillation.

9. The method as claimed in claim 1, characterized in that the first and the second deflection or deflection rate is in each case determined in the form of the resultant of the oscillation.

10. (canceled)

11. (canceled)

12. (canceled)

13. The method as claimed in claim 1, characterized in that the tissue has a shear modulus μ1 at the first point in time and a shear modulus μ2 at the second point in time and their ratio with respect to one another is determined on the basis of the first amplitude A1 determined at the first point in time and the second amplitude A2 determined at the second point in time according to the following equation:
$$\frac{{μ}_{2}}{{μ}_{1}} = {( \frac{{A}_{1}}{{A}_{2 }} )}^{4}$$

14. The method as claimed in claim 1, characterized in that the deflections or deflection rates of the wave are determined at a number of points in time in order to determine a time profile of the deflection or the deflection rate.

15. The method as claimed in claim 1, characterized in that the first and the second amplitude of the deflection or the deflection rate of the oscillation are determined by means of a correlation between the time profile of the deflection or deflection rate and a harmonic oscillator function.

16. The method as claimed in claim 15, characterized in that the harmonic oscillator function has an oscillation frequency which corresponds to the frequency of the wave excited in the tissue.

17. The method as claimed in claim 1, characterized in that the first and the second deflection or deflection rate of the excited wave is determined using magnetic resonance imaging, wherein a phase signal depending on the deflection or deflection rate of the oscillation is determined, and the phase signal is determined at a number of points in time such that a time profile of the phase signal results from which a time profile of the deflection or deflection rate of the wave excited in the tissue can be determined.

18. (canceled)

19. The method as claimed in claim 10, characterized in that the time profile of the deflection or deflection rate is correlated to a harmonic oscillator function, wherein the harmonic oscillator function has an oscillation frequency which corresponds to the frequency of the wave excited in the tissue.

20. The method as claimed in claim 10, characterized in that a time profile of the amplitude of the deflection and/or of the amplitude of the deflection rate is determined from the time profile of the phase signal.

21. The method as claimed in claim 12, characterized in that an amplitude A of the wave excited in the tissue by the frequency ω is determined at different times t from the deflection rate \dot{u} of the oscillation as follows:
$$A ( t ) = \frac{1}{N π} \vert {\int }_{t}^{t + Δ t} \dot{u} exp ( iω τ ) d τ \vert ;$$
$$with$$
$$Δ t = N \cdot \frac{2 π}{ω} , N = 1 , 2 , 3 , …$$

22. The method as claimed in claim 12, characterized in that an amplitude A of the wave excited in the tissue by the frequency ω is determined at different times t from the deflection u of the oscillation as follows:
$$A ( t ) = \frac{ω}{N π} \vert {\int }_{t}^{t + Δ t} u exp ( iω τ ) d τ \vert ;$$
$$with$$
$$Δ t = N \cdot \frac{2 π}{ω} , N = 1 , 2 , 3 , …$$

23. (canceled)

24. (canceled)

25. An apparatus for the elastographic examination of tissue, with
deflection determination means for determining a deflection and/or deflection rate of mechanical waves excited in the tissue which oscillate predominately or exclusively transversely with respect to the direction of propagation thereof, wherein
the tissue has first elastic properties at a first point in time and second elastic properties at a second point in time, which second elastic properties differ from the first elastic properties; and
the deflection determination means are designed and provided to determine a first deflection or deflection rate at the first point in time and a second deflection or deflection rate at the second point in time.

26. (canceled)

27. (canceled)

28. (canceled)

29. (canceled)

30. (canceled)
